(19) United States
(12) Patent Application Publication
(10) Pub. No.: US 2011/0300618 A1
(43) Pub. Date: Dec. 8, 2011

(54) SEMI-AUTOMATED DEVICE FOR SINGLE PARAMETER AND MULTI-PARAMETER PHENOTYPING OF CELLS

(76) Inventors: Joel Lieblein, Sudbury, MA (US); Ronald Lasser, Andover, MA (US); Dan Pankowsky, Franklin, TN (US); Jill Gershon, Franklin, TN (US); Daniel Jones, Concord, MA (US)

(21) Appl. No.: 13/081,593

(22) Filed: Apr. 7, 2011

Related U.S. Application Data
(60) Provisional application No. 61/321,583, filed on Apr. 7, 2010.

(51) Int. Cl. C12M 1/34

(52) U.S. Cl. 435/287.1

(57) ABSTRACT

A single-use device that produces a readily interpretable, visual presentation of biomaterial, enabling single parameter and multi-parameter characterizing of said biomaterial by means of visual inspection, particularly for immunophenotyping. In one embodiment, the device uses antibody coated microspheres which are adapted to bind to specific types of cells. An arrangement of uniquely molded components, which may be plastic or other suitable material, are assembled so as to provide a sequential procedure capable of incubating a suspension comprised of antibody coated microspheres, biomaterial, and liquid suspension agents. Following incubation, the invention further removes liquids from said suspension, and provides for rinsing the incubated retentate and for removing said rinsing liquids from the incubated retentate. The invention further provides for affixing the incubated retentate onto a standard glass microscope slide to enable phenotyping of incubated biomaterial (e.g., cells) by means of visual inspection or other analyses that can be performed from a glass slide.

This application claims benefit of and priority to U.S. Provisional Application No. 61/321,583, filed Apr. 7, 2010, and is entitled to that filing date for priority. The specification, figures and complete disclosure of U.S. Provisional Application No. 61/321,583 are incorporated herein by specific reference for all purposes.

### FIELD OF INVENTION

This invention relates to a device for the single parameter and multi-parameter characterizing of biomaterial, such as cells.

### BACKGROUND OF THE INVENTION

The ability to characterize multiple cellular characteristics of a target cell population continues to grow in importance. This importance arises from demands in both clinical and research biomedical applications to be able to subdivide and understand specific cell populations. This is particularly true in the treatment of cancers, as there is an increasing awareness of the importance of genetic variations between different patients of apparently the same apparent tumor type. In the past, this demand was met by using a combination of different technologies particularly flow cytometry and a variety of cytogenetic and molecular procedures. But at the same time, the amount of tissue available to the laboratory diagnostician or the researcher has shrunk as operative techniques have been developed to allow analysis of ever decreasing sample sizes. The requirements for the future will be to extract more information from less tissue. Therefore, it will become necessary to combine methodologies in a useful way which can be referred to as multiparameter phenotypic analysis.

Antibody coated microspheres, referred to as “spheres”, “microspheres”, or “beads,” offer an inert solid support on which to perform a variety of different laboratory analyses. The binding of such beads to intact cells was pioneered by Hajek and Russell (U.S. Pat. No. 5,554,505) and Fostad, et al. (U.S. Pat. No. 6,265,229), which are incorporated herein in their entireties by specific reference for all purposes. However, these methodologies did not lend themselves to be easily adapted for routine laboratory use. This issue was addressed by Pankowsky (U.S. Pat. Nos. 6,682,940; 6,828,157; 7,351,589; 7,354,773; and 7,364,906), which are incorporated herein in their entireties by specific reference for all purposes. In these patents, Pankowsky taught cell-bead complexes formed in a cell suspension are filtered and transferred to a glass slide. Using a glass slide as the support media to perform additional analysis makes a wide range of sophisticated laboratory techniques available. This innovation makes multiparameter phenotypic analysis available in any laboratory, not just specialized centers. Since microspheres are inert and can be easily identified, they can serve as markers of the presence of certain cell types. At the same time, since they are inert, they do not interfere with other desired laboratory analysis. So a target cell population is bound to beads, enriched by filtration, and then printed on to the glass slide. The presence of the beads allows for surface phenotyping of the cells. The application of additional stains allows for either morophologic examination and/or the identification of additional cellular characteristics. If methods such as in-situ hybridization is applied to the glass slide, then a target cell population can be simultaneously phenotyped and genotyped.

However, the previous Pankowsky inventions required manual laboratory techniques that made the final results susceptible to individual operator techniques that could ruin the final results. The process was entirely manual and required delicate manipulation of the sample.

### SUMMARY OF INVENTION

In various exemplary embodiments, the present invention provides a new and useful, single-use device that produces a readily interpretable, visual presentation of biomaterial (e.g., cells), enabling single parameter and multi-parameter characterizing of said biomaterial by means of visual inspection, particularly for immunophenotyping. In one embodiment, the present invention, as shown in FIGS. 1-8, uses antibody coated microspheres which are adapted to bind to specific types of cells. An arrangement of uniquely molded components, which may be plastic or other suitable material, are assembled so as to provide a sequential procedure capable of incubating a suspension comprised of antibody coated microspheres, biomaterial, and liquid suspension agents. Following incubation, the invention further removes liquids from said suspension, and provides for rinsing the incubated retentate and for removing said rinsing liquids from the incubated retentate. The invention further provides for affixing the incubated retentate onto a standard glass microscope slide to enable phenotyping of incubated biomaterial (cells) by means of visual inspection or other analyses that can be performed from a glass slide.

In one embodiment, the set of components are assembled so as to provide a series of internal latching interlocks. The invention further provides a means for the user to easily alter the arrangement of said interlocks, enabling the user to initiate a series of precisely controlled rearrangements of the plastic components by means of sequentially arranging the interlocks and providing a force at an exterior portion of the device. The invention further provides for precisely controlling forces that may be applied by the user.

In another exemplary embodiment, an enclosed, transparent incubation chamber is molded of a material whose totality of intermolecular forces is non-attractive to the antibody coated microspheres and whose base is comprised of a suspended hydrophobic membrane with a uniform pore diameter that is not larger than the diameter of the smallest antibody coated microsphere and whose pore diameter is sufficient to prevent capillary penetration by the liquid suspension agents. The incubation chamber is arranged with a self-sealing septum at an exterior portion of the device. A suspension comprised of antibody coated microspheres, biomaterial, and liquid suspension agents are created within the transparent incubation chamber by sequential injection via the self-sealing septum.

The transparent incubation chamber, being held in position via the compression of a resilient member, is so formed as to move axially along a precise path per the arrangement of the interlocks and the application of a force by the user to bring the external surface of the suspended hydrophobic membrane into contact with an array of absorbent fibers sufficient to compel a capillary action of the liquid suspension agents through the suspended hydrophobic membrane. The invention further provides for the precise opposite motion of the transparent incubation chamber per arrangement of the interlocks by means of the force of the resilient member. Subsequent injection of additional liquid agents and the removal of said liquid agents may be accomplished by means of the same precise movements of the transparent incubation chamber per the arrangement of the interlocks and the application of a force by the user at an exterior portion of the device.

The suspended hydrophobic membrane, being positioned via the interlocks and the compression of a resilient member, is so formed to move axially along a precise path when so directed by the user to bring the interior surface of the suspended hydrophobic membrane into contact with a standard glass microscope slide. Precise opposite motion may be accomplished per positioning of the interlocks of the suspended hydrophobic membrane by means of the force of the resilient member. The invention further provides for the positioning by the user of the device's molded components for subsequent removal of the standard microscope slide from the device to enable visual inspection, and further provides for permanently sealing liquid agents within the device by means of positioning of the interlocks and via a force applied by the user. Permanent alteration of internal components can be accomplished by positioning of the interlocks so as to prevent possible reuse of the device.

### DESCRIPTION OF THE DRAWINGS

FIG. 1 shows a front perspective view of a device in accordance with an exemplary embodiment of the present invention.

FIG. 2 shows a rear perspective view of the device of FIG. 1.

FIG. 3 shows a top view of the device of FIG. 1.

FIG. 4 shows a front view of the device of FIG. 1.

FIG. 5 shows a rear view of the device of FIG. 1.

FIG. 6 shows a bottom view of the device of FIG. 1.

FIG. 7 shows a right side view of the device of FIG. 1.

FIG. 8 shows a left side view of the device of FIG. 1.

### DETAILED DESCRIPTION OF EXEMPLARY EMBODIMENTS

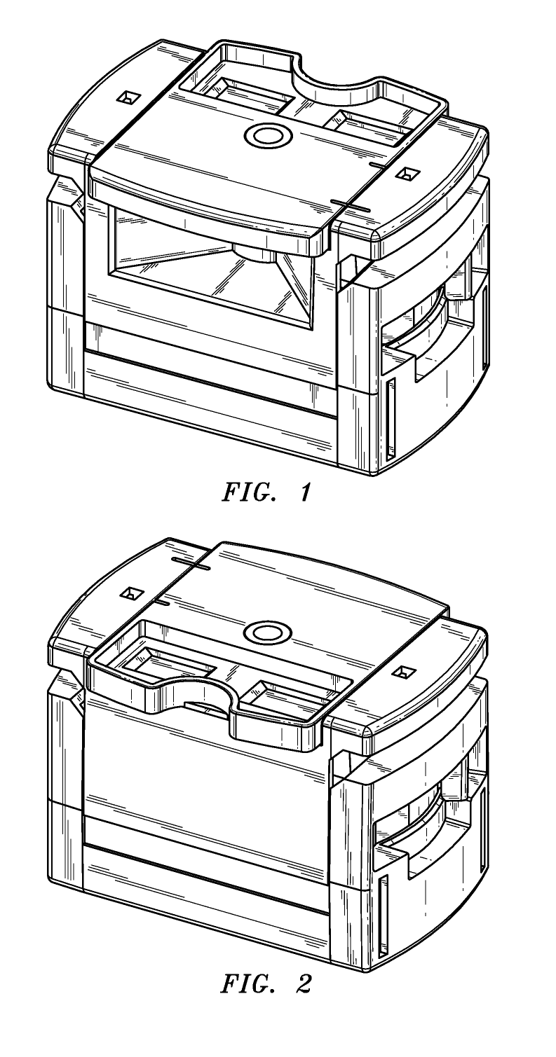

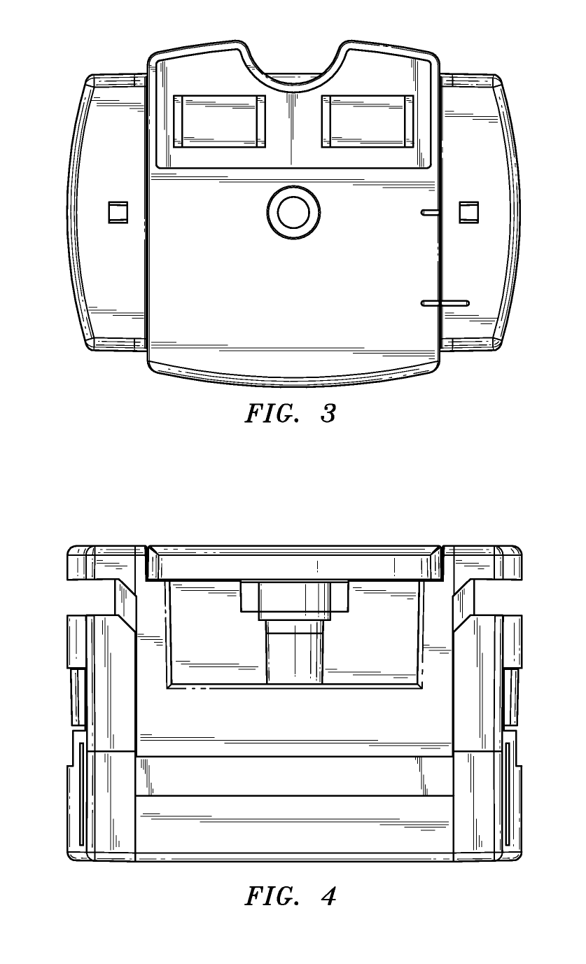

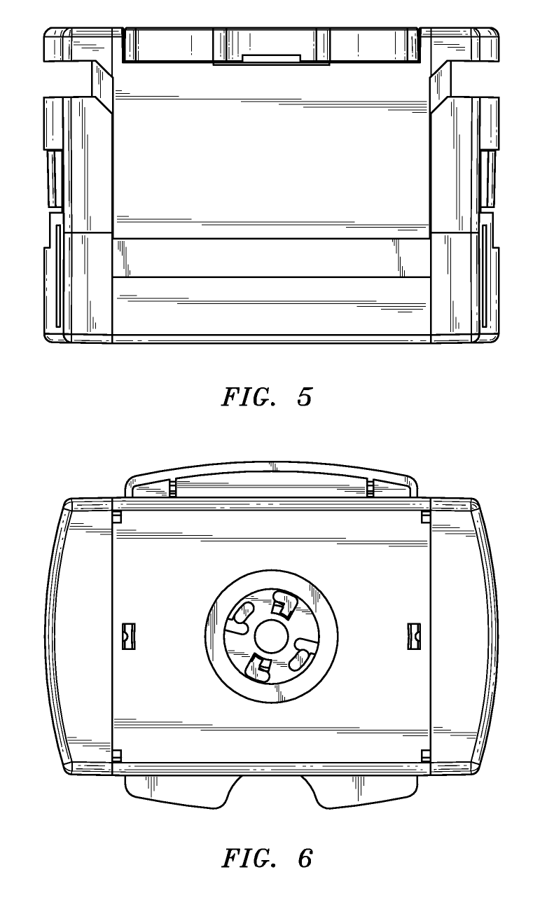

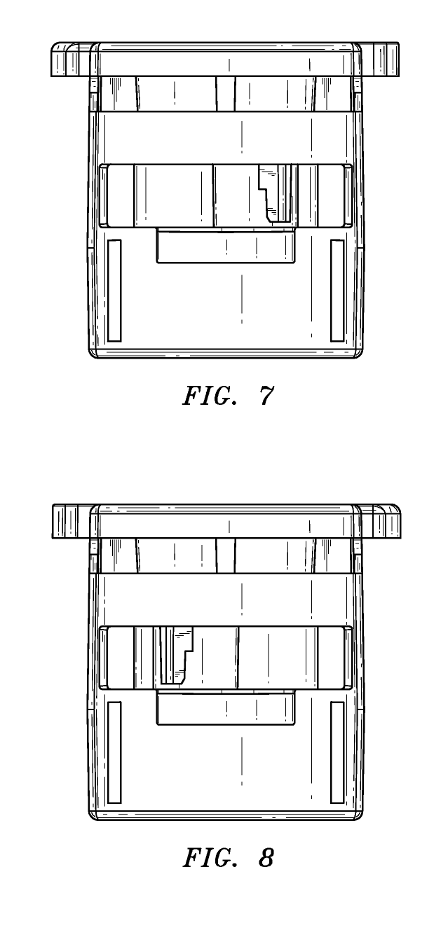

In various exemplary embodiments, the present invention provides a new and useful, single-use device that produces a readily interpretable, visual presentation of biomaterial (e.g., cells), enabling single parameter and multi-parameter characterizing of said biomaterial by means of visual inspection, particularly for immunophenotyping. In one embodiment, the present invention, as shown in FIGS. 1-8, uses antibody coated microspheres which are adapted to bind to specific types of cells. An arrangement of uniquely molded components, which may be plastic or other suitable material, are assembled so as to provide a sequential procedure capable of incubating a suspension comprised of antibody coated microspheres, biomaterial, and liquid suspension agents. Following incubation, the invention further removes liquids from said suspension, and provides for rinsing the incubated retentate and for removing said rinsing liquids from the incubated retentate. The invention further provides for affixing the incubated retentate onto a standard glass microscope slide to enable phenotyping of incubated biomaterial (cells) by means of visual inspection or other analyses that can be performed from a glass slide.

In one embodiment, the set of components are assembled so as to provide a series of internal latching interlocks. The invention further provides a means for the user to easily alter the arrangement of said interlocks, enabling the user to initiate a series of precisely controlled rearrangements of the plastic components by means of sequentially arranging the interlocks and providing a force at an exterior portion of the device. The invention further provides for precisely controlling forces that may be applied by the user.

In another exemplary embodiment, an enclosed, transparent incubation chamber is molded of a material whose totality of intermolecular forces is non-attractive to the antibody coated microspheres and whose base is comprised of a suspended hydrophobic membrane with a uniform pore diameter that is not larger than the diameter of the smallest antibody coated microsphere and whose pore diameter is sufficient to prevent capillary penetration by the liquid suspension agents. The incubation chamber is arranged with a self-sealing septum at an exterior portion of the device. A suspension comprised of antibody coated microspheres, biomaterial, and liquid suspension agents are created within the transparent incubation chamber by sequential injection via the self-sealing septum.

The transparent incubation chamber, being held in position via the compression of a resilient member, is so formed as to move axially along a precise path per the arrangement of the interlocks and the application of a force by the user to bring the external surface of the suspended hydrophobic membrane into contact with an array of absorbent fibers sufficient to compel a capillary action of the liquid suspension agents through the suspended hydrophobic membrane. The invention further provides for the precise opposite motion of the transparent incubation chamber per arrangement of the interlocks by means of the force of the resilient member. Subsequent injection of additional liquid agents and the removal of said liquid agents may be accomplished by means of the same precise movements of the transparent incubation chamber per the arrangement of the interlocks and the application of a force by the user at an exterior portion of the device.

The suspended hydrophobic membrane, being positioned via the interlocks and the compression of a resilient member, is so formed to move axially along a precise path when so directed by the user to bring the interior surface of the suspended hydrophobic membrane into contact with a standard glass microscope slide. Precise opposite motion may be accomplished per positioning of the interlocks of the suspended hydrophobic membrane by means of the force of the resilient member. The invention further provides for the positioning by the user of the device's molded components for subsequent removal of the standard microscope slide from the device to enable visual inspection, and further provides for permanently sealing liquid agents within the device by means of positioning of the interlocks and via a force applied by the user. Permanent alteration of internal components can be accomplished by positioning of the interlocks so as to prevent possible reuse of the device.

Accordingly, embodiments of the present invention comprise a machine that automates the crucial steps of the Pankowsky process, as described in Pankowsky (U.S. Pat. Nos. 6,682,940; 6,828,157; 7,351,589; 7,354,773; and 7,364,906), which are incorporated herein in their entireties by specific reference for all purposes. This makes the makes the final product reproducible and is independent of variations introduced by any particular individual.

Thus, it should be understood that the embodiments and examples described herein have been chosen and described in order to best illustrate the principles of the invention and its practical applications to thereby enable one of ordinary skill in the art to best utilize the invention in various embodiments and with various modifications as are suited for particular uses contemplated. Even though specific embodiments of this invention have been described, they are not to be taken as exhaustive. There are several variations that will be apparent to those skilled in the art.

What is claimed is:

1. A device for preparing incubated biomaterial, comprising:
an enclosed incubation chamber with a self-sealing septum and a base comprising a suspended hydrophobic membrane with an internal surface and an external surface;
wherein the incubation chamber, after sequential injection of biomaterial, antibody-coated microspheres and liquid suspension agents into the incubation chamber through the self-sealing septum, may be moved along a precise path to bring an array of absorbent fibers into contact with the external surface of the suspended hydrophobic membrane sufficient to compel capillary action of the liquid suspension agents through the membrane.

2. The device of claim 1, further wherein the suspended hydrophobic membrane may subsequently be moved along a precise path to bring the interior surface of the suspended hydrophobic membrane into contact with a glass slide.

3. The device of claim 1, further comprising a resilient compression member positioned to hold the enclosed incubation chamber in its original position.

4. The device of claim 1, wherein the incubation chamber is moved by application of an external force.

5. The device of claim 1, wherein the components of the device are assembled to provide a series of internal latching interlocks.

6. The device of claim 1, wherein the device is single use.
